(19) United States
(12) Patent Application Publication
(10) Pub. No.: US 2009/0023631 A1
(43) Pub. Date: Jan. 22, 2009

(54) Composition and Method of Use for Soft Tissue Augmentation/Drug Delivery

(76) Inventors: Z. Paul Lorenc, New York, NY (US); Robert C. Di Luccio, Haymarket, VA (US)

Correspondence Address: RYAN, MASON & LEWIS, LLP, 90 FOREST AVENUE, LOCUST VALLEY, NY, 11560, US

(21) Appl. No.: 12/174,769

(22) Filed: Jul. 17, 2008

Related U.S. Application Data
(60) Provisional application No. 60/950,423, filed on Jul. 18, 2007.

(51) Int. Cl. A61K 38/28; A61K 38/18

(52) U.S. Cl. 514/3; 514/12

(57) ABSTRACT

A composition for augmenting soft tissue and drug delivery is provided. The composition includes an active ingredient, a carrier, and a cross-linking agent, wherein the carrier is cross-linked with the cross-linking agent and the active ingredient is combined with the cross-linked carrier into a combination having a therapeutic effect. Techniques are also provided for producing a composition for augmenting soft tissue. Also, techniques are provided for soft tissue augmentation. Additionally, techniques are also provided for delivering a composition.

### CROSS-REFERENCE TO RELATED APPLICATION(S)

This application claims the benefit of U.S. Provisional Application Ser. No. 60/950,423, filed on Jul. 18, 2007, the disclosure of which is incorporated by reference herein.

### FIELD OF THE INVENTION

The present invention generally relates to biology, and, more particularly, to soft tissue augmentation.

### BACKGROUND OF THE INVENTION

Along with a trend toward living a longer, healthier life, more and more patients are looking to physicians to help them achieve a more youthful appearance. The skin's natural aging process manifests contour changes and rhytids secondary to the depletion of subcutaneous fat and the loss of dermal collagen.

Traditionally, rejuvenation has been achieved with a face-lift by surgically tightening the skin. Today, a multitude of minimally invasive procedures are aimed at rejuvenation without the risk, recovery time, and expense of major surgery. The development and popularity of BOTOX® has opened the door for equally noninvasive, adjunctive treatment of dynamic rhytids and soft tissue augmentation.

Soft tissue augmentation has become a popular means of addressing issues such as, for example, contour defects that result from aging, photo-damage, trauma and/or scarification, or disease. A number of filling agents exist in the armamentarium. Therefore, the physician is responsible for knowing which substance is best suited to address a particular defect and the patient or disease process.

### SUMMARY OF THE INVENTION

Principles of the present invention provide compositions and methods of use for soft tissue augmentation and drug delivery. An exemplary composition for augmenting soft tissue, according to one aspect of the invention, can include an active ingredient, a carrier, and a cross-linking agent, wherein the carrier is cross-linked with the cross-linking agent and the active ingredient is combined with the cross-linked carrier into a combination having a therapeutic effect.

An exemplary method for producing a composition for augmenting soft tissue, according to one aspect of the invention, can include the steps of obtaining an active ingredient, obtaining a carrier, cross-linking the carrier with a cross-linking agent, and combining the active ingredient and the cross-linked carrier into a combination having a therapeutic effect.

An exemplary method for soft tissue augmentation, according to one aspect of the invention, can include administering a composition to a patient, wherein the composition includes a combination of an active ingredient and a carrier cross-linked with a cross-linking agent. Additionally, an exemplary method for delivering a composition, according to one aspect of the invention, can include the steps of cross-linking a carrier with a cross-linking agent, incorporating an active ingredient into the cross-linked carrier, and using the cross-linked carrier to deliver the active ingredient to a target site.

These and other objects, features and advantages of the present invention will become apparent from the following detailed description of illustrative embodiments thereof.

### DETAILED DESCRIPTION OF PREFERRED EMBODIMENTS

Principles of the present invention introduce including growth factors (such as tissue growth factor, angiogenesis and transforming growth factor) into cross-linked hyaluronic acid NASHA-type products to directly stimulate the formation of collagen through the stimulation of wound repair mechanisms described earlier, and to therefore stimulate fibroblasts so that the appearance of new connective tissue is evident and a significantly prolonged improvement is seen in the appearance of treated skin and/or correction of contour deformities. Additionally, an introduction of hormones, polypeptides or other proteins into the cross-linked hyaluronic acid NASHA-type products may be performed and serve as a vehicle for drug, hormone, and/or protein delivery.

By way of example, as used herein, “soft tissue” refers to any tissue supporting, surrounding or connecting structures of the body of a patient. Also, by way of example, as used herein, “soft tissue augmentation” refers to any volume amplification of soft tissue including, but not limited to, cosmetic, medical or reconstructive procedures.

By way of further example, a “therapeutic effect” in a treatment methodology may be defined herein as an effect sufficient to produce a measurable increase in soft tissue augmentation in the patient and/or a prolonged effect and increased collagen production in comparison to existing approaches (for example, an improvement of up to about 50%). The term “patient” as used herein is intended, for example, to refer broadly to mammalian subjects, preferably humans receiving medical attention (for example, diagnosis, monitoring, etc.), care or treatment.

It has been realized that the processes that lead to wound repair and collagen deposition that can help promote longevity of a filler are three major processes: the inflammatory phase, the proliferative phase, and the maturational phase.

The inflammatory phase is characterized by hemostasis and inflammation. Collagen exposed during wound formation activates the clotting cascade (both the intrinsic and extrinsic pathways), initiating the inflammatory phase. After injury to tissue occurs, the cell membranes, damaged from the wound formation, release thromboxane A2 and prostaglandin 2-alpha, potent vasoconstrictors. This initial response helps to limit hemorrhage. After a short period, capillary vasodilatation occurs secondary to local histamine release, and the cells of inflammation are able to migrate to the wound bed. The timeline for cell migration in a normal wound healing process is predictable.

Platelets, the first response cell, release multiple chemokines, including epidermal growth factor (EGF), fibronectin, fibrinogen, histamine, platelet-derived growth factor (PDGF), serotonin, and von Willebrand factor. These factors help stabilize the wound through clot formation. These mediators act to control bleeding and limit the extent of injury. Platelet degranulation also activates the complement cascade, specifically C5a, which is a potent chemo-attractant for neutrophils.

The inflammatory phase continues, and more immune response cells migrate to the wound. The second response cell to migrate to the wound, the neutrophil, is responsible for debris scavenging, complement-mediated opsonization of bacteria, and bacteria destruction via oxidative burst mechanisms (that is, superoxide and hydrogen peroxide formation). The neutrophils kill bacteria and decontaminate the wound from foreign debris.

The next cells present in the wound are leukocytes and macrophages (monocytes). The macrophage, also referred to as the orchestrator, is essential for wound healing. Numerous enzymes and cytokines are secreted by the macrophage. These include, for example, collagenases (which debride the wound), interleukins and tumor necrosis factor (TNF) (which stimulate fibroblasts (produce collagen) and promote angiogenesis), and transforming growth factor (TGF) (which stimulates keratinocytes). This step marks the transition into the process of tissue reconstruction, that is, the proliferative phase.

The second stage of wound healing is the proliferative phase. Epithelialization, angiogenesis, granulation tissue formation, and collagen deposition are the principal steps in this anabolic portion of wound healing. Epithelialization occurs early in wound repair. If the basement membrane remains intact, the epithelial cells migrate upwards in the normal pattern. This is equivalent to a first-degree skin burn. The epithelial progenitor cells remain intact below the wound, and the normal layers of epidermis are restored in 2-3 days. If the basement membrane has been destroyed, similar to a second- or third-degree burn, then the wound is re-epithelialized from the normal cells in the periphery and from the skin appendages, if intact (for example, hair follicles, sweat glands).

Angiogenesis, stimulated by TNF-alpha, is marked by endothelial cell migration and capillary formation. The new capillaries deliver nutrients to the wound and help maintain the granulation tissue bed. The migration of capillaries into the wound bed is critical for proper wound healing. The granulation phase and tissue deposition require nutrients supplied by the capillaries, and failure for this to occur results in a chronically unhealed wound. Mechanisms for modifying angiogenesis are under study and have significant potential to improve the healing process.

The final part of the proliferative phase is granulation tissue formation. Fibroblasts differentiate and produce ground substance and then collagen. The ground substance is deposited into the wound bed, and collagen is then deposited as the wound undergoes the final phase of repair. Many different cytokines are involved in the proliferative phase of wound repair. The steps and the exact mechanism of control have not been elucidated. Some of the cytokines include, for example, PDGF, insulin-like growth factor (IGF), and EGF.

The final phase of wound healing is the maturational phase. The wound undergoes contraction, ultimately resulting in a smaller amount of apparent scar tissue. The entire process is a dynamic continuum with an overlap of each phase and continued remodeling. The wound reaches maximal strength at one year, with a tensile strength that is 30% of normal skin. Collagen deposition continues for a prolonged period, but the net increase in collagen deposition plateaus after 21 days.

In existing approaches, cytokines have a limited role in clinical practice. For example, the only currently available commercial product proven to be efficacious in randomized, double blind-studies is PDGF, available as recombinant human PDGF-BB. In multiple studies, recombinant human PDGF-BB has been demonstrated to reduce healing time and improve the incidence of complete wound healing in stage III and IV ulcers.

Proper wound healing involves a complex interaction of cells and cytokines working in concert. In recent years, more chemical mediators integral to this process have been identified, however the sequential steps and specific processes have not been fully differentiated.

Soft tissue augmentation can be produced with the introduction of active ingredients with existing marked products or autologous tissues isolated either through liposuction or other means. Aside from products that are being practiced based on autologous tissue, biological products or synthetic materials, existing approaches are unable to achieve the longevity desired by today's patient. Consequently, a need exists to be able to deliver fillers (that is, substances that enhance, augment, fill or add to an existing or lacking volume) to soft tissues and to prolong the longevity of the fillers by stimulating growth and cell reproduction, while at the same time maintaining the desired corrected effect. Soft-tissue fillers, which can include, for example, injectable collagen, fat or hyaluronic acid (HA), can help fill in lines and creases that are a consequence of the aging process, temporarily restoring a smoother, more youthful-looking appearance. When injected beneath the skin, fillers plump up creased and sunken areas of the face. They can also add fullness to the lips and cheeks. Injectable fillers may be used alone or in conjunction with a resurfacing procedure, such as a laser treatment, or a recontouring procedure, such as a facelift.

As noted above, there is a need to introduce a filler that can take advantage of the immediate restorative affects of agents such as hyaluronic acid and prolong their effectiveness and lifetime. An example of such is the introduction of active ingredients that can stimulate cells to produce collagen, elastin and stimulate cellular growth.

Investigators have shown that the introduction of growth hormone (GH) from polymeric blends such as collagen and hyaluronic acid release physiological concentrations of growth hormone. As such, GH influences cellular growth, specifically stimulation of human osteoblast-like cells (HOBs). The researchers measured this affect by getting a sense of cell proliferation and alkaline phosphatase (ALP), which is a biochemical marker of HOB, thereby concluding that cell differentiation is possible. Carriers for these hormones have improved with time, as products such as, for example, Restylane are cross-linked to prolong their lifetime when injected as a filler. For example, in an existing approach, a NASHA version of hyaluronic acid is produced from a microorganism Streptococcus zooepidemicus to produce highly pure hyaluronic acid that is chemically identical to that that exists in the skin and other tissues.

Existing techniques such as, for example, U.S. Pat. No. 5,827,937, have been established to cross-link hyaluronic acid (HA). It is possible to introduce GH into cross-linked versions of HA in situ and to utilize the hydrogel as a carrier and stabilizer for the hormone. In existing approaches other than those cross-linking versions of hyaluronic acid, it has been shown that HA/Pluronic composite hydrogels could be formed demonstrating thermally reversible swelling, de-swelling behaviors which could regulate the release of rhGH (recombinant human growth hormone). Sustained release of the hormone has been observed from these composite HA structures. Other cross-linking strategies of hyaluronic acid have also been studied as scaffolds, wherein a cross-linking strategy targeting the alcohol groups via a poly(ethylene glycol) diepoxide cross-linker was investigated for the generation of degradable HA hydrogels. For additional support for cell adhesion in vitro, collagen was incorporated into the HA solution prior to the cross-linking process. Additionally, using biotinylation enables one to fashion molecules that have the ability to attach avidin-type biomolecules.

Other existing approaches have investigated the use of both CRS (Cimicifuga racemosa Extract) and TNS (Tissue Nutrient Solution Recovery Complex (TNS) (SkinMedica, Carlsbad, Calif., USA), a product containing a variety of growth factors including VEGF, PDGF-A, G-CSF, HGF, IL-6, IL-8, and TGF-b1) provide significant improvement in the appearance of facial rhytides. The other ingredients in CRS alone, minus the TGF-b1, however, failed to demonstrate a statistically significant improvement in rhytides. It is suggested that if the growth factor portion of the product stimulates neocollagenesis and collagen remodeling, then the incorporation of supplemental L-ascorbic acid, a necessary cofactor for collagen synthesis, could enhance this process.

Another existing study demonstrated that the injection of cross-linked hyaluronic acid stimulates collagen synthesis. The investigators determined that by injecting a hyaluronic acid, NASHA-type product, Restylane, they were able to observe intracellular and extracellular detection of type 1 procollagen synthesis in the skin treated with the NASHA product because this is a pattern that is consistent with the production of type 1 collagen, from fibroblasts. Additionally, the authors noted the stimulation of tissue growth factor and transforming growth factorβ.

As noted above, numerous filling agents or fillers exist. Examples may include the following items. Autologous collagen (fat) works by promoting an inflammatory response that, in turn, results in the deposition of new collagen at the recipient site. Harvested autologous fat from procedures such as, for example, liposuction, is processed by mixing it with sterile distilled water and then allowing it to freeze, thereby leading to the rupture of adipocytes. The liquefied fraction of intracellular triglycerides is then ready to be injected through a fine-gauge needle (for example, a 30-gauge needle) suitable for intradermal injection. This technique is often used in conjunction with subcutaneous fat transplantation.

The benefit of this technique is its autologous nature, negating the need for hypersensitivity testing. However, it requires harvesting the tissue from a donor site as well as involved preparation and expedient administration after harvesting the tissue. Also, studies vary on the duration of autologous collagen. The range is months to years, depending on the methods used in fat harvesting, processing, and transplanting.

Restylane is an FDA-approved non-animal-stabilized hyaluronic acid derivative used for soft tissue augmentation. Unlike Hylaform gel, it is derived from streptococcal bacterial fermentation and does not require an animal source. At 20 milligrams per milliliter (mg/mL), Restylane has a higher concentration of hyaluronic acid than Hylaform gel. It is used to treat rhytids and scars, and is also used in lip augmentation. Restylane correction was noted to be 82% at 3 months and 33% at 1 year in a study involving 285 wrinkles treated in 113 patients.

Perlane is also a hyaluronic acid derivative at 20 mg/mL, but it is a more robust form of Restylane for use in the deep dermis and at the dermal-fat junction. It is used in Canada and has received FDA approval in 2007.

Being hyaluronic acid derivatives similar to Hylaform Gel, Restylane and Perlane have less risk of clumping and can be inserted more smoothly. Overcorrection is not needed with these products.

Sculptra is poly-L-lactic acid and is FDA-approved for the treatment of HIV facial lipoatrophy. It serves as a volume enhancer and is used for indications similar to those for autologous fat transfer. Results are not immediate. Treatment is performed as a series of 3-5 treatments approximately one month apart.

New-fill is the name under which Sculptra is marketed in most countries outside the United States. New-fill is a non-animal-derived polylactic acid touted to be biocompatible, biodegradable, and immunologically inert. It is distributed freeze-dried, can be stored at room temperature, and is reconstituted with sterile water. New-fill is injected either into the superficial dermis for the treatment of rhytids and acne scars or subdermally to treat lipodystrophy of the cheeks and hands, liposuction contour deformities, and lip atrophy.

However, all the above existing products and approaches lack longevity associated with administration of each product. Principles of the present invention introduce an active ingredient to promote the longevity of such a product by stimulating appropriate cytokines known in wound repair such as, for example, optimizing the proliferative phase of wound repair when there is granulation tissue formation.

As described herein, fibroblasts differentiate, produce ground substance and then collagen. The ground substance is deposited into the wound bed. Collagen is then deposited as the wound undergoes the final phase of repair. Many different cytokines are involved in the proliferative phase of wound repair. Some of the cytokines include, for example, PDGF, insulin like growth factor (IGF), and EGF.

Additionally, stimulating angiogenesis with redeposited fat cells may be advantageous by using of interleukins and tumor necrosis factor (TNF), which stimulate fibroblasts (produce collagen) and promote angiogenesis, and transforming growth factor (TGF), which stimulates keratinocytes. This, consequently, would lead to the step that marks the transition into the process of tissue reconstruction, that is, the proliferative phase, as described above.

Principles of the present invention include a composition and corresponding method of use for soft tissue augmentation, wherein the composition includes a filler cross-linked with a cross-linking agent, which is combined with an active ingredient. In one or more embodiments, a cross-linking agent includes a sugar, such as ribose, and an active ingredient includes a growth factor. Also, a sugar used as a cross-linking agent can include, for example, a naturally occurring reducing sugar such as diose, a triose, a tetrose, a pentose, a hexose, a septose, an octose, a nanose, or a decose. A sugar may also include, for example, glycerose, threose, erythrose, lyxose, xylose, arabinose, ribose, allose, altrose, glucose, mannose, gulose, idose, galactose and talose. Additionally, a sugar can also be in the form of a disaccharide such as maltose, lactose, sucrose, cellobiose, gentiobiose, melibiose, turanose, and trehalose.

Additionally, an active ingredient may also include insulin or one or more hormones. As described herein, cross-linked HA has the ability to incorporate active proteins and peptides that may have usefulness in drug delivery applications and therefore provide a unique opportunity to deliver these agents in a matrix that would allow for sustained delivery of the active ingredient.

One or more embodiments of the present invention develop formulations for Transforming Growth Factor (TGF). There are two classes of TGFs that are not structurally or genetically related to one another, and they act through different receptor mechanisms. The two types of Transforming Growth Factors are as described below.

TGFα is upregulated in some human cancers. It is produced in macrophages, brain cells, and keratinocytes, and induces epithelial development. TGFβ, the class of TGF used in connection with principles of the present invention, exists in three known subtypes in humans: TGFβ1, TGFβ2, and TGFβ3. These subtypes are upregulated in some human cancers, and play crucial roles in tissue regeneration, cell differentiation, embryonic development, and regulation of the immune system.

TGFβ receptors are single pass serine/threonine kinase receptors. These proteins were originally characterized by their capacity to induce oncogenic transformation in a specific cell culture system, namely rat kidney fibroblasts. Application of the transforming growth factors to normal rat kidney fibroblasts induces cultured cells to proliferate and overgrow, no longer subject to the normal inhibition caused by contact between cells.

One or more embodiments of the present invention use other classes of growth factors such as, for example, epidermal growth factor (EGF), fibroblast growth factor (FGF2), nerve growth factor and platelet-derived growth factor.

A preferred embodiment of the present invention uses a filler commonly known as hyaluronan (also called hyaluronic acid or hyaluronate), which is a non-sulfated glycosaminoglycan distributed widely throughout connective, epithelial, and neural tissues. Hyaluronan is one of the chief components of the extracellular matrix; it contributes significantly to cell proliferation and migration, and may also be involved in the progression of some malignant tumors. The average 70 kilogram (kg) man has roughly 15 grams of hyaluronan in his body, one third of which is turned over (that is, degraded and synthesized) every day.

Hyaluronan is naturally found in many tissues of the body such as, for example, skin, cartilage, and the vitreous humor. It is therefore well suited to biomedical applications targeting these tissues. Hyaluronan biomedical products have been approved for use in, for example, eye surgery (that is, corneal transplantation, cataract surgery, glaucoma surgery and surgery to repair retinal detachment) and ophthalmic surgery.

Hyaluronan is also used to treat osteoarthritis of the knee. Such treatments are administered as a course of injections into the knee joint and are believed to supplement the viscosity of the joint fluid, thereby lubricating the joint, cushioning the joint and producing an analgesic effect. It has also been suggested that hyaluronan has positive biochemical effects on cartilage cells. Recently, oral use of hyaluronan has been suggested although effectiveness needs to be demonstrated. Some preliminary clinical studies exist that suggest that oral administration of hyaluronan has a positive effect on osteoarthritis.

Due to its high biocompatibility and its common presence in the extracellular matrix of tissues, hyaluronan is gaining popularity as a biomaterial scaffold in tissue engineering research. Additionally, in some cancers, hyaluronan levels correlate well with malignancy and poor prognosis. Hyaluronan is thus often used as a tumor marker for prostate and breast cancer. It may also be used to monitor the progression of the disease.

Hyaluronan may also be used postoperatively to induce tissue healing, notably after cataract surgery. Current models of wound healing propose that larger polymers of hyaluronic acid appear in the early stages of healing to physically make room for white blood cells, which in turn mediate the immune response. One or more embodiments of the present invention include fillers such as, for example, collagen, fat, alginates, gelatin, collagen-based fillers and poly-L-lactic acid.

Principles of the invention include combining a growth factor and a carrier/filler (for example, hyaluronan) so that the active ingredient is delivered in a pharmacologically active state. In a preferred embodiment, the target concentration of the active ingredient is in a range of from about 50 nanograms per milliliter (ng/ml) to about one mg/ml.

The combination of the active ingredient (for example, a growth factor) with the carrier component (for example, hyaluronan) can be done through the use of aqueous or non-aqueous solvents that will not denature the protein. Once combined, delivery of the composition may be accomplished, for example, intracutaneous, subcutaneous, intramuscular, or as a bolus.

Principles of the invention include advantages over existing approaches such as, for example, potential stability of the protein in the formulation, increased longevity of effectiveness, increased effectiveness, and localized delivery.

As described herein, one or more embodiments of the invention include a composition for augmenting soft tissue, wherein the composition includes an active ingredient, a carrier, and a cross-linking agent, wherein the carrier is cross-linked with the cross-linking agent and the active ingredient is combined with the cross-linked carrier into a combination having a therapeutic effect. The active ingredient can include a growth factor (such as for example, transforming growth factor-β (TGFβ), epidermal growth factor (EGF), fibroblast growth factor (FGF2), nerve growth factor and/or platelet-derived growth factor).

The active ingredient can include, for example, insulin and/or hormones. Also, as noted herein, the active ingredient can have a concentration in a range of from about 50 nanograms per milliliter (ng/ml) to about one milligram per milliliter (mg/ml). The carrier can include, for example, hyaluronan, and the cross-linking agent can include a sugar. Exemplary sugars can include ribose, glycerose, threose, erythrose, lyxose, xylose, arabinose, ribose, allose, altrose, glucose, mannose, gulose, idose, galactose, talose, a naturally occurring reducing sugar (for example, a diose, a triose, a tetrose, a pentose, a hexose, a septose, an octose, a nanose, and/or a decose), and/or a disaccharide, (for example, maltose, lactose, sucrose, cellobiose, gentiobiose, melibiose, turanose, and/or trehalose).

Further, the active ingredient can be combined with the cross-linked carrier via use of aqueous solvents and/or non-aqueous solvents.

The techniques described herein also include producing a composition for augmenting soft tissue including the steps of obtaining an active ingredient, obtaining a carrier, cross-linking the carrier with a cross-linking agent, and combining the active ingredient and the cross-linked carrier into a combination having a therapeutic effect.

Additionally, one or more embodiments of the invention include techniques for soft tissue augmentation including administering a composition (for example, a therapeutically effective amount of a composition) to a patient, wherein the composition includes a combination of an active ingredient and a carrier cross-linked with a cross-linking agent. Administering a composition to a patient can include at least one of administering a composition to a patient intracutaneously, subcutaneously, intramuscularly and/or as a bolus.

Additionally, an exemplary method for delivering a composition, according to one aspect of the invention, can include the steps of cross-linking a carrier with a cross-linking agent, incorporating an active ingredient into the cross-linked carrier, and using the cross-linked carrier to deliver the active ingredient to a target site. The active ingredient can include, for example a protein, a peptide and/or a growth factor.

Below are three illustrative examples of active ingredients that can be included in the matrix of hyaluronic acid that can be cross-linked by incubation with the active ingredient. These examples only illustrate a method that can be employed to incorporate the active ingredients.

In an exemplary embodiment, hyaluronic acid (HA) can be prepared with TGF-β as follows. In a standard 3 neck 500 ml organic reaction kettle with a safe-lab stirrer bearing, 1 millimole (mmole) of sodium hyaluronate (HA), 1.1 mMoles ribose were added to water to form a 1% solution. The HA and ribose were completely dissolved in the water, resulting in the formation of a viscous liquid adjusted to a pH of 6 to 7. To that solution, about 0.1% weight/weight (w/w) (based on solids), TGF is added and stirred.

The above solution is incubated at 37° C. to allow for association of the sugar, growth factor and hyaluronic acid anywhere from 2 hours to 200 hours. This results in a composition that can be further processed to a formulation that can be freeze-dried. The resulting mixture may be freeze-dried to yield a mixture of the HA and the active ingredient. This can then be reconstituted for injection.

In another exemplary embodiment, hyaluronic acid (HA) can be prepared with insulin as follows. In a standard 3 neck 500 ml organic reaction kettle with a safe-lab stirrer bearing. 1 mMole of sodium hyaluronate (HA), 1.1 mMoles ribose were added to water to form a 1% solution. The HA and ribose were completely dissolved in the water, resulting in the formation of a viscous liquid with a pH of 6 to 7. To that solution, 1000 units of insulin is added and stirred.

The above solution is incubated at 37° C. to allow for association of the sugar, growth factor and hyaluronic acid anywhere from 2 hours to 200 hours. This results in a composition that can be further processed to a formulation that can be freeze-dried. The resulting mixture may be freeze-dried to yield a mixture of the HA and the active ingredient. This can then be reconstituted for injection.

Additionally, yet another exemplary embodiment can use all of the elements of the two examples above, except that in lieu of incubation, the active ingredient and the HA-ribose may be exposed to low levels of eBeam radiation or ultraviolet light to promote cross-linking of the composition with the active ingredient. In this situation, less than 1 megaelectron volt (MeV) may be utilized at very short duration of 10 seconds or less.

Alternatively, an ultraviolet irradiating device (3 kilowatt (kW) metal halide lamp) may be used for less than five minutes of exposure insuring the solution never exceeds 37° C. This can be repeated until the desired level of cross-linking is achieved.

Further, the techniques described here can also include the use of resveratrol (a phytoalexin found, for example, in grapes and other plants). By way of example, the amount of resveratrol in food can vary, as wine, for example, contains between 0.2 and 40 milligrams per liter (mg/L) of resveratrol.

One or more embodiments of the present invention also include incorporating adipose-derived stem cells in a HA lattice and/or matrix. Autologous fat can be harvested from a patient and adipose-derived stem cells can be extracted therefrom. By way of example, about 250 cubic centimeters (CCs) of autologous fat can be harvested from a patient, and up to 75×10⁶ adipose-derived stem cells can be extracted from the autologous fat. Additionally, up to 75×10⁶ adipose-derived stem cells can be incorporated into, for example, 1 to 10 CCs of hyaluronic acid (HA) lattice.

At least one embodiment of the invention may provide one or more beneficial effects, such as, for example, stimulating appropriate cytokines known in wound repair such as, for example, optimizing the proliferative phase of wound repair when there is granulation tissue formation.

Although illustrative embodiments of the present invention have been described herein, it is to be understood that the invention is not limited to those precise embodiments, and that various other changes and modifications may be made by one skilled in the art without departing from the scope or spirit of the invention.

What is claimed is:

1. A composition for augmenting soft tissue, wherein the composition comprises:
an active ingredient;
a carrier; and
a cross-linking agent, wherein the carrier is cross-linked with the cross-linking agent and the active ingredient is combined with the cross-linked carrier into a combination having a therapeutic effect.

2. The composition of claim 1, wherein the active ingredient comprises a growth factor.

3. The composition of claim 2, wherein the growth factor comprises transforming growth factor-β (TGFβ).

4. The composition of claim 2, wherein the growth factor comprises at least one of epidermal growth factor (EGF), fibroblast growth factor (FGF2), nerve growth factor and platelet-derived growth factor.

5. The composition of claim 1, wherein the active ingredient comprises at least one of insulin and one or more hormones.

6. The composition of claim 1, wherein the active ingredient comprises a concentration in a range of from about 50 nanograms per milliliter (ng/ml) to about one milligram per milliliter (mg/ml).

7. The composition of claim 1, wherein the carrier comprises hyaluronan.

8. The composition of claim 1, wherein the cross-linking agent comprises a sugar.

9. The composition of claim 8, wherein the sugar comprises at least one of ribose, glycerose, threose, erythrose, lyxose, xylose, arabinose, ribose, allose, altrose, glucose, mannose, gulose, idose, galactose, talose, a naturally occurring reducing sugar, wherein the naturally occurring reducing sugar comprises at least one of a diose, a triose, a tetrose, a pentose, a hexose, a septose, an octose, a nanose, and a decose, and a disaccharide, wherein the disaccharide comprises at least one of maltose, lactose, sucrose, cellobiose, gentiobiose, melibiose, turanose, and trehalose.

10. The composition of claim 1, wherein the active ingredient is combined with the cross-linked carrier via use of at least one of one or more aqueous solvents and one or more non-aqueous solvents.

11. A method for producing a composition for augmenting soft tissue, comprising the steps of:
obtaining an active ingredient;
obtaining a carrier;
cross-linking the carrier with a cross-linking agent; and
combining the active ingredient and the cross-linked carrier into a combination having a therapeutic effect.

12. The method of claim 11, wherein the active ingredient comprises a growth factor.

13. The method of claim 12, wherein the growth factor comprises transforming growth factor-β (TGFβ).

14. The method of claim 11, wherein the carrier comprises hyaluronan.

15. The method of claim 11, wherein the cross-linking agent comprises a sugar.

16. A method for soft tissue augmentation, comprising administering a composition to a patient, wherein the composition comprises a combination of an active ingredient and a carrier cross-linked with a cross-linking agent.

17. The method of claim 16, wherein the active ingredient comprises a growth factor.

18. The method of claim 17, wherein the growth factor comprises transforming growth factor-β (TGFβ).

19. The method of claim 16, wherein the carrier comprises hyaluronan.

20. The method of claim 16, wherein the cross-linking agent comprises a sugar.

21. The method of claim 16, wherein administering a composition to a patient comprises at least one of administering a composition to a patient intracutaneously, administering a composition to a patient subcutaneously, administering a composition to a patient intramuscularly and administering a composition to a patient as a bolus.

22. A method of delivering a composition, comprising the steps of:
cross-linking a carrier with a cross-linking agent;
incorporating an active ingredient into the cross-linked carrier; and
using the cross-linked carrier to deliver the active ingredient to a target site.

23. The method of claim 22, wherein the active ingredient comprises at least one of a pharmaceutical agent, a drug, a protein, a peptide, insulin, one or more hormones, one or more adipose-derived stem cells and a growth factor.

24. The method of claim 22, wherein the carrier comprises hyaluronan.

25. The method of claim 22, wherein the cross-linking agent comprises a sugar.
